(19) United States
(12) Patent Application Publication
(10) Pub. No.: US 2023/0114835 A1
(43) Pub. Date: Apr. 13, 2023

(54) CONTROLLING CYTOCHROME C OXIDASE OF A LIGHT SOURCE BASED ON UV IRRADIATION AMOUNT

(71) Applicant: SIGNIFY HOLDING B.V., EINDHOVEN (NL)

(72) Inventors: BIANCA MARIA IRMA VAN DER ZANDE, HEEZE (NL); REMY CYRILLE BROERSMA, EINDHOVEN (NL); TOBIAS BORRA, RIJSWIJK (NL); MARCUS THEODORUS MARIA LAMBOOIJ, EINDHOVEN (NL)

(21) Appl. No.: 17/909,870

(22) PCT Filed: Mar. 3, 2021
(86) PCT No.: PCT/EP2021/055297

§ 371 Date: Sep. 7, 2022

(30) Foreign Application Priority Data
Mar. 12, 2020 (EP) 20162572.0

(51) Int. Cl. A61N 5/06

(52) U.S. Cl. CPC A61N 5/0616; A61N 2005/0628; A61N 2005/0659; A61N 2005/0661; A61N 2005/0663

(57) ABSTRACT

A method comprises determining (203) whether a person has been and/or will be irradiated with an amount of UV radiation exceeding a threshold and controlling (205), in dependence on the determination, one or more light sources to render light comprising a light component having wavelengths in the range 550 to 900 nm. A spectral power distribution of the light is chosen such that the light has a cytochrome C oxidase efficacy complying with the following conditions: for DNA synthesis: the cytochrome C oxidase efficacy is at least 6.2*vʹ-2.48 mW/lm if the light’s vʹ is lower than 0.539 or at least 0.85 mW/lm if the light’s vʹ is equal to or higher than 0.539; for RNA synthesis: the cytochrome C oxidase efficacy is at least 7.5*vʹ-2.975 mW/lm if the light’s vʹ is lower than 0.539 or at least 1.05 mW/lm if the light’s vʹ is equal to or higher than 0.539.

### FIELD OF THE INVENTION

The invention relates to a system for controlling one or more light sources to render light comprising a light component having wavelengths in the range 550 to 900 nm.

The invention further relates to a method of controlling one or more light sources to render light comprising a light component having wavelengths in the range 550 to 900 nm.

The invention also relates to a computer program product enabling a computer system to perform such a method.

### BACKGROUND OF THE INVENTION

People (young and old) nowadays spend more than 90% indoors and often in urban areas where sunlight cannot reach the ground due to the densely built-up areas. Therefore, the indoor environment becomes paramount for people’s health and wellbeing. Moreover, healthy building design seems to become more and more an agenda point for building owners, regulation bodies and tenants. Consequently, the industry trend towards artificial lighting schemes that reflect the earth’s natural light-dark cycle as well as the introduction of access to daylight develops a market pull for general solutions to provide the light nutrition required to support health and well-being. Light recipes might become enablers for (context aware) healthy indoor environments.

Light is the most efficient trigger to regulate vitamin D related processes in the body and as such a better solution to beat the vitamin D deficiency than the supplements currently available. Vitamin D is a hormone regulating numerous cell functions that control our health and wellbeing. Vitamin D deficiency is for instance linked to not only bone strength, but also coronary heart diseases, infectious diseases, neuropsychiatric diseases, diabetes II and some cancers. 90% of our Vitamin D comes from sunlight (UV-B).

However, people spent 90% of their times indoors, resulting in too little UV exposure. Already, 60-90% of the population report deficient and insufficient serum vitamin D levels. Artificial light is able to deliver the estimated amounts of UV-B to reduce vitamin D deficiency. However, crossing the safety threshold for erythemal action will introduce a health risk related to skin cancer.

US 2006/0184214 A1 describes the use of naturally derived or artificially created or genetically engineered photolyase enzymes or related enzymes or other proteins for DNA or RNA repair. However, the use of these enzymes is often not sufficient to decrease the risk of UV-B irradiation to a person’s health sufficiently.

### SUMMARY OF THE INVENTION

It is a first object of the invention to provide a system, which helps decrease the risk of UV-B irradiation to a person’s health.

It is a second object of the invention to provide a method, which helps decrease the risk of UV-B irradiation to a person’s health.

In a first aspect of the invention, a system for controlling one or more light sources to render light comprising a light component having wavelengths in the range 550 to 900 nm comprises at least one control interface and at least one processor configured to determine whether a person has been and/or will be irradiated with an amount of UV radiation exceeding a threshold based on at least one of an amount of UV radiation received by a light sensor and UV radiation information from a control signal to the lighting device, and control, via said at least one control interface, in dependence on said determination, said one or more light sources to render light comprising a light component having wavelengths in the range 550 to 900 nm, a spectral power distribution of said light being chosen such that said light has a cytochrome C oxidase efficacy complying with one or more of the following conditions (i) the cytochrome C oxidase efficacy for DNA synthesis is at least (6.2*vʹ-2.48) mW/lm if said light’s vʹ is lower than 0.539 or is at least 0.85 mW/lm if said light’s vʹ is equal to or higher than 0.539, and (ii) the cytochrome C oxidase efficacy for RNA synthesis is at least (7.5*vʹ-2.975) mW/lm if said light’s vʹ is lower than 0.539 or is at least 1.05 mW/lm if said light’s v' is equal to or higher than 0.539, wherein the cytochrome C oxidase efficacy is defined as:

$$\begin{matrix}C y t o c h r o m e C o x i d a s e E f f i c a c y o f r a d i a t i o n \left(W / L m\right) = \\ \frac{{\int }_{550}^{900} {Φ}_{e , λ} \left(λ\right) {s}_{c y t} \left(λ\right) d λ}{683 {\int }_{380}^{780} {Φ}_{e , λ} \left(λ\right) V \left(λ\right) d λ}\end{matrix}$$

wherein:
Φ_{e,λ}(λ) = is said spectral power distribution of said light
s_{cyt}(λ) = is the spectral sensitivity of cytochrome C oxidase activation for either DNA synthesis or RNA synthesis;
V(λ) = is the photopic luminosity function; and
v′ is a color coordinate of said light in the CIE 1976 Uniform Chromaticity Scale diagram. In optimized conditions, the skin can repair UV induced damage by itself
when the cellular ATP (energy) levels of the skin cells are freely available and high. This free energy can be achieved with a cytochrome C oxidase efficacy of radiation that is sufficiently high, especially through deep red/near infrared (NIR) light stimulation. However, the content of deep red and NIR light in the current electrical light sources for general illumination, especially in fluorescent and LED light sources, is minimal. This may be due to an ever increasing desire to maximize luminous efficacy.

As a result, many humans have a limited deep red/NIR stimulation and adding high energy light such as UV(-B) radiation can therefore be potentially harmful for skin. By rendering light with a cytochrome C oxidase efficacy of radiation that is sufficiently high when a person has been and/or will be irradiated with an amount of UV radiation exceeding a threshold, skin damage may be prevented and/or repaired. Thus, a light source that can activate the preventing/repairing capacity of the skin may be used to help reduce the potential skin cancer risk of UV-B irradiation.

M. Fitzgerald et al. describe in “Red/near-infrared irradiation therapy for treatment of central nervous system injuries and disorders”, Rev. Neurosci. 2013;24(2):205-26. doi: 10.1515/revneuro-2012-0086, amongst others that there is “strong evidence that 670-nm irradiation gives significant protection to the retina, and some studies indicate that cytochrome c oxidase is the most likely photoreceptor”, but this only pertains to protection to the retina and is unrelated to UV-B irradiation.

The most pronounced and efficient Cytochrome C oxidase activation seems to be in the wavelengths between 550-900 nm. Said light component preferably comprises wavelengths in the range 600 to 850 nm, even more preferably wavelengths in at least one of the ranges: 605 to 635 nm, 660 to 690 nm, 755 to 790 nm and 800 to 835 nm. These wavelengths have a relatively high cytochrome C oxidase activation for DNA synthesis and/or RNA synthesis. The peak wavelength of the light may be one of these wavelengths.

Said at least one processor may be configured to control, via said at least one control interface, said one or more light sources to render said light during one or more periods that start at most 24 hours before said UV radiation and end at most 24 hours after said UV radiation. By rendering the light during these one or more periods, the chance of preventing and/or repairing skin damage is highest.

Said at least one processor may be configured to control, via said at least one control interface, said one or more light sources to render said light such that said light includes at least part of said UV radiation, said UV radiation being rendered with a minimum standard erythemal dose of 0.01 per day and a maximum standard erythemal dose of 10 per day. By letting the system control both the rendering of the light component having wavelengths in the range 550 to 900 nm and the UV radiation, typically having wavelengths in the range 280 to 315 nm (UV-B) and/or in the range 315-400 nm (UV-A), it may be possible to ensure that no UV radiation is rendered without skin damage preventing and/or repairing light being also rendered. This system may be a therapy device, for example. The therapy device may be intended or suitable for psoriasis treatment, for example. A minimum standard erythemal dose of 0.01 per day is typically necessary to start vitamin D production and maximizing the rendered UV radiation to a standard erythemal dose of 10 per day helps decrease the risk of the UV irradiation to a person’s health.

Said at least one processor may be configured to determine an amount of UV radiation received by a light sensor and determine whether said person has been irradiated with an amount of UV radiation exceeding said threshold based on said determined amount of UV radiation received by said light sensor. This makes it possible to determine how much artificial UV radiation has been rendered by another lighting system without receiving this information from this other lighting system and makes it possible to determine how much UV radiation has been received from the sun.

Said at least one processor may be configured to determine a minimum target value for said cytochrome C oxidase efficacy based on a desired color coordinate vʹ such that said minimum target value for DNA synthesis is at least 6.2*vʹ-2.48 mW/lm if said desired color coordinate vʹ is lower than 0.539 or at least 0.85 mW/lm if said desired color coordinate vʹ is equal to or higher than 0.539 and said minimum target value for RNA synthesis is at least 7.5*vʹ-2.975 mW/lm if said desired color coordinate vʹ is lower than 0.539 or at least 1.05 mW/lm if said desired color coordinate vʹ is equal to or higher than 0.539, choose said spectral power distribution of said light such that said light comprises a light component having wavelengths in the range 550 to 900 nm, said desired color coordinate vʹ is achieved and said cytochrome C oxidase efficacy of said light has a value which equals or exceeds said minimum target value. This allows a system in which the user can select a light color setting of his choosing to determine a suitable spectral power distribution based on this desired color.

Said at least one processor may be configured to determine said minimum target value for said cytochrome C oxidase efficacy further based on said amount of UV radiation. A higher than normal minimum target value for the cytochrome C oxidase efficacy may be used to increase the chance of preventing and/or repairing skin damage when the amount of UV exposure is higher than normal.

Said light may comprise further light components which make said light look white. This allows the preventing and/or repairing light to be provided by a general illuminating device. A separate lighting device for repairing and/or preventing skin damage may therefore not be necessary.

Said at least one processor may be configured to control said one or more light sources to render said light component in a pulsating manner. Alternatively, said at least one processor may be configured to control said one or more light sources to render said component continuously.

In a second aspect of the invention, a method of controlling one or more light sources to render light comprising a light component having wavelengths in the range 550 to 900 nm comprises determining whether a person has been and/or will be irradiated with an amount of UV radiation exceeding a threshold based on at least one of an amount of UV radiation received by a light sensor and UV radiation information from a control signal from a transmitter to the lighting device, and controlling, in dependence on said determination, said one or more light sources to render light comprising a light component having wavelengths in the range 550 to 900 nm, a spectral power distribution of said light being chosen such that said light has a cytochrome C oxidase efficacy complying with one or more of the following conditions (i) the cytochrome C oxidase efficacy for DNA synthesis is at least (6.2*vʹ-2.48)mW/lm if said light’s vʹ is lower than 0.539 or is at least 0.85 mW/lm if said light’s vʹ is equal to or higher than 0.539, and (ii) the cytochrome C oxidase efficacy for RNA synthesis is at least (7.5*vʹ-2.975) mW/lm if said light’s vʹ is lower than 0.539 or is at least 1.05 mW/lm if said light’s vʹ is equal to or higher than 0.539, wherein the cytochrome C oxidase efficacy is defined as:

$$\begin{matrix}C y t o c h r o m e C o x i d a s e E f f i c a c y o f r a d i a t i o n \left(W / L m\right) = \\ \frac{{\int }_{550}^{900} {Φ}_{e , λ} \left(λ\right) {s}_{c y t} \left(λ\right) d λ}{683 {\int }_{380}^{780} {Φ}_{e , λ} \left(λ\right) V \left(λ\right) d λ}\end{matrix}$$

wherein:
Φ_{e,λ}(λ) = is said spectral power distribution of said light
s_{cyt}(λ) = is the spectral sensitivity of cytochrome C oxidase activation for either DNA synthesis or RNA synthesis;
V(λ) = is the photopic luminosity function; and
vʹ is a color coordinate of said light in the CIE 1976 Uniform Chromaticity Scale diagram.

It is particularly noted that said method counteracts and/or prevents damage to the skin of humans.

Said method may be performed by software running on a programmable device. This software may be provided as a computer program product.

Moreover, a computer program for carrying out the methods described herein, as well as a non-transitory computer readable storage-medium storing the computer program are provided. A computer program may, for example, be downloaded by or uploaded to an existing device or be stored upon manufacturing of these systems.

A non-transitory computer-readable storage medium stores at least one software code portion, the software code portion, when executed or processed by a computer, being configured to perform executable operations for controlling one or more light sources to render light comprising a light component having wavelengths in the range 550 to 900 nm.

The executable operations comprise determining whether a person has been and/or will be irradiated with an amount of UV radiation exceeding a threshold and controlling, in dependence on said determination, said one or more light sources to render light comprising a light component having wavelengths in the range 550 to 900 nm, a spectral power distribution of said light being chosen such that said light has a cytochrome C oxidase efficacy complying with one or more of the following conditions (i) the cytochrome C oxidase efficacy for DNA synthesis is at least (6.2*vʹ-2.48)mW/lm if said light’s vʹ is lower than 0.539 or is at least 0.85 mW/lm if said light’s vʹ is equal to or higher than 0.539, and (ii) the cytochrome C oxidase efficacy for RNA synthesis is at least (7.5*vʹ-2.975) mW/lm if said light’s vʹ is lower than 0.539 or is at least 1.05 mW/lm if said light’s vʹ is equal to or higher than 0.539, wherein the cytochrome C oxidase efficacy is defined as:

$$\begin{matrix}C y t o c h r o m e C o x i d a s e E f f i c a c y o f r a d i a t i o n \left(W / L m\right) = \\ \frac{{\int }_{550}^{900} {Φ}_{e , λ} {s}_{c y t} \left(λ\right) d λ}{683 {\int }_{380}^{780} {Φ}_{e , λ} \left(λ\right) V \left(λ\right) d λ}\end{matrix}$$

wherein:
Φ_{e,λ}(λ) = is said spectral power distribution of said light
s_{cyt}(λ) = is the spectral sensitivity of cytochrome C oxidase activation for either DNA synthesis or RNA synthesis;
V(λ) = is the photopic luminosity function; and
vʹ is a color coordinate of said light in the CIE 1976 Uniform Chromaticity Scale diagram.

As will be appreciated by one skilled in the art, aspects of the present invention may be embodied as a device, a method or a computer program product. Accordingly, aspects of the present invention may take the form of an entirely hardware embodiment, an entirely software embodiment (including firmware, resident software, microcode, etc.) or an embodiment combining software and hardware aspects that may all generally be referred to herein as a “circuit”, “module” or “system.” Functions described in this disclosure may be implemented as an algorithm executed by a processor/microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present invention, a computer readable storage medium may be any tangible medium that can contain, or store, a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java(TM), Smalltalk, C++ or the like and conventional procedural programming languages, such as the “C” programming language or similar programming languages. The program code may execute entirely on the user’s computer, partly on the user’s computer, as a stand-alone software package, partly on the user’s computer and partly on a remote computer, or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or a central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of devices, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

### BRIEF DESCRIPTION OF THE DRAWINGS

These and other aspects of the invention are apparent from and will be further elucidated, by way of example, with reference to the drawings, in which:

FIG. 1 is a block diagram of a first embodiment of the system;

FIG. 2 is a block diagram of a second embodiment of the system;

FIG. 3 is a block diagram of a third embodiment of the system;

FIG. 4 shows a first example of UV radiation and visible light being rendered over time;

FIG. 5 shows a second example of UV radiation and visible light being rendered over time;

FIG. 6 shows a third example of UV radiation and visible light being rendered over time;

FIG. 7 shows a fourth example of UV radiation and visible light being rendered over time;

FIG. 8 is a flow diagram of a first embodiment of the method;

FIG. 9 is a flow diagram of a second embodiment of the method; and

FIG. 10 is a block diagram of an exemplary data processing system for performing the method of the invention.

Corresponding elements in the drawings are denoted by the same reference numeral.

### DETAILED DESCRIPTION OF THE EMBODIMENTS

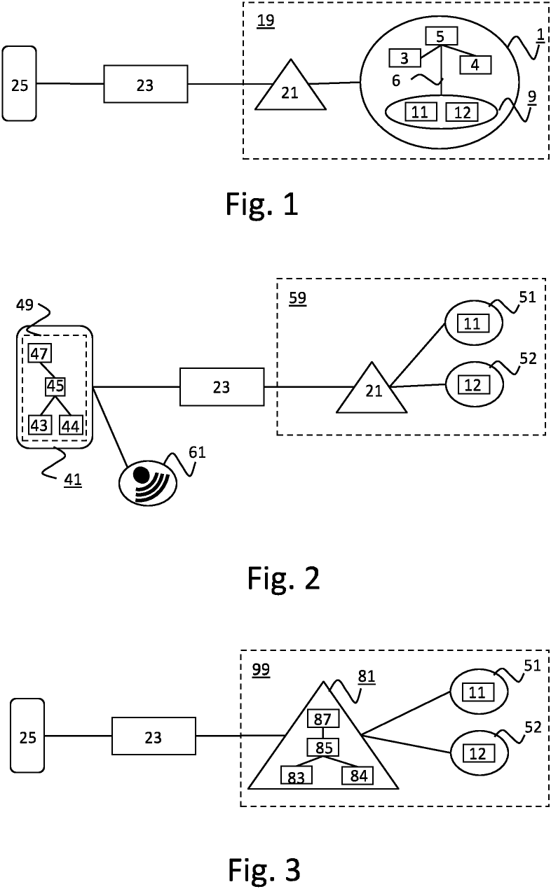

FIG. 1 shows a first embodiment of the system for controlling one or more light sources to render light comprising a light component having wavelengths in the range 550 to 900 nm: a lighting device 1. The lighting device 1 comprises a receiver 3, a transmitter 4, a processor 5, a LED module 9 and a control interface 6 between the processor 5 and the LED module 9. The LED module 9 comprises a plurality of LEDs: a visible-light LED 11 and an UV-B LED 12.

The processor 5 is configured to determine whether a person has been and/or will be irradiated with an amount of UV radiation exceeding a threshold and control, via the control interface 5, in dependence on the determination, the visible-light LED 11 (e.g. a red LED) of the LED module 9 to render light comprising a light component having wavelengths in the range 550 to 900 nm. The light component preferably comprises wavelengths in the range 600 to 850 nm, e.g. in the range 605 to 635 nm, in the range 660 to 690 nm, in the range 755 to 790 nm and/or in the range 800 to 835 nm. The light component may be a red component, for example.

A spectral power distribution of the light is chosen such that the light has a cytochrome C oxidase efficacy complying with one or more of the following conditions:
(i) the cytochrome C oxidase efficacy for DNA synthesis is at least (6.2*vʹ-2.48) mW/lm if the light’s vʹ is lower than 0.539 or is at least 0.85 mW/lm if the light’s vʹ is equal to or higher than 0.539, and
(ii) the cytochrome C oxidase efficacy for RNA synthesis is at least (7.5*vʹ-2.975) mW/lm if the light’s vʹ is lower than 0.539 or is at least 1.05 mW/lm if the light’s vʹ is equal to or higher than 0.539,
wherein the cytochrome C oxidase efficacy (also referred to as “CC_eff”) is defined as:

$$\begin{matrix}C y t o c h r o m e C o x i d a s e E f f i c a c y o f r a d i a t i o n \left(W / L m\right) = \\ \frac{{\int }_{550}^{900} {Φ}_{e , λ} {s}_{c y t} \left(λ\right) d λ}{683 {\int }_{380}^{780} {Φ}_{e , λ} \left(λ\right) V \left(λ\right) d λ}\end{matrix}$$

wherein:
Φ_{e,λ}(λ) = is the spectral power distribution of the light
s_{cyt}(λ) = is the spectral sensitivity of cytochrome C oxidase activation for either DNA synthesis or RNA synthesis;
V(λ) = is the photopic luminosity function; and
vʹ is a color coordinate of the light in the CIE 1976 Uniform Chromaticity Scale diagram.

In the embodiment of FIG. 1, the processor 5 is configured to control, via the control interface 6, the UV-B LED 12 of the LED module 9 to render the light such that the light includes at least part of the UV radiation. The UV radiation is rendered with a minimum standard erythemal dose of 0.01 per day and a maximum standard erythemal dose of 10 per day and comprises wavelengths in the range 280 to 315 nm.

The UV radiation can be rendered with a minimum standard erythemal dose (SED) of 0.01 per day and a maximum standard erythemal dose (SED) of 10 per day by having the UV light source(s) irradiate the person with an energy between 1 and 1000 Joules per m². This may be achieved by rendering UV light at a higher power for a shorter duration or at a lower power for a longer duration. There are multiple ways of designing/making a lighting device which is able to achieve an irradiance (Watt per m²) sufficient to provide an energy between 1 and 1000 Joules per m² in a day. The power at which the UV light source(s) need(s) to render the UV light normally depends on the distance between the UV light source(s) and the person.

The UV radiation may be rendered by a light source similar to the one disclosed in US 2006/0184214, for example. The UV radiation is preferably rendered with a standard erythemal dose (SED) that depends on a person’s skin type, since whether erythema is attained with a certain dose of UV radiation depends on the person’s skin type. The system may determine that if it controls a light source to render UV radiation with a dose that attains erythema, the person will be irradiated with an amount of UV radiation exceeding the threshold.

The light may comprise further light components which make the light look white. The term white light relates to light having a correlated color temperature (CCT) between about 2000 K and 20000 K and within about 10 to 15 SDCM (standard deviation of color matching) from the BBL (black body locus).

A mobile device 25 is able to control the lighting device 1 via a wireless LAN access point 23 and a bridge 21, e.g. with the help of a light control app running on the mobile device 25. A user of the mobile device 25 may be able to change a color and/or intensity of the visible light and/or start and stop UV irradiation, for example. In the embodiment of FIG. 1, the mobile device 25 and the lighting device 1 communicate via the bridge 21. In an alternative embodiment, the mobile device 25 and the lighting device 1 can communicate directly, e.g. using Bluetooth technology. A lighting system 19 comprises the lighting device 1 and the bridge 21.

Presence detection may be used to avoid unnecessary energy consumption by switching off one or more of the LEDs 11-12 when no one is present in the room or when a specific person is not at his desk.

The LEDs 11-12 may be direct emitting or phosphor converted LEDs. The visible-light LED 11 may be a red LED, for example. In the embodiment of FIG. 1, the LED module 9 comprises only one visible-light LED 11. In an alternative embodiment, the LED module 9 comprises multiple visible-light LEDs, e.g. a red LED, a green LED, a blue LED and optionally a white LED. In the embodiment of FIG. 1, the LED module 9 comprises only one UV-B LED 12. In an alternative embodiment, the LED module 9 comprises multiple UV-B LEDs.

The lighting device 1 may further comprise a multi-channel driver and the processor 5 may be part of a lighting controller. The controller may be able to vary light output depending on the time of day, season and or individual and as such matching the circadian needs of humans. For example, the cytochrome C may be above a certain value in the morning (preventive) and/or in the evening (repairing) while UV-B output is induced in between.

Four examples of special distributions created with a combination of a white LED and an IR LED, and further using luminescent material, are shown in Table 1. The desired and achieved cytochrome C oxidase efficacy is listed in mW/lm. In all four examples, vʹ is lower than 0.539 and the desired cytochrome C oxidase efficacy is therefore at least (6.2*vʹ-2.48)mW/lm for DNA synthesis and at least (7.5*vʹ-2.975) mW/lm for RNA synthesis. In all four examples, the achieved cytochrome C oxidase efficacy exceeds the desired cytochrome C oxidase efficacy for both DNA and RNA synthesis.

TABLE 1

Examples of spectral distributions

| Ex | Description | vʹ | uʹ | Desired CC_eff DNA | Desired CC_eff RNA | Achieved CC_eff DNA | Achieved CC_eff RNA |
|---|---|---|---|---|---|---|---|
| 1 | 450 nm LED + (Y,Lu)AG:Ce + (Ba,Sr,Ca)AlSiN₃:Eu + 765 nm LED | 0.527 | 0.263 | 0.79 | 0.98 | 0.98 | 1.14 |
| 2 | 450 nm LED + (Y,Lu)AG:Ce + Mn⁴⁺ phosphor + 674 nm LED + 764 nm LED | 0.4755 | 0.205 | 0.47 | 0.59 | 0.95 | 1.01 |
| 3 | 450 nm LED + (Y,Lu)AG:Ce + Mn⁴⁺ phosphor + 674 nm LED | 0.4758 | 0.216 | 0.47 | 0.59 | 0.67 | 0.75 |
| 4 | 450 nm LED + (Y,Lu)AG:Ce + (Ba,Sr,Ca)AlSiN₃:Eu + 674 nm LED | 0.4655 | 0.2005 | 0.41 | 0.52 | 0.85 | 0.92 |

The photopic sensitivities for wavelengths in the range 380 to 780 nm are listed in Table 2:

TABLE 2

photopic human eye sensitivities

| λ | Photopic sensitivity | λ | Photopic sensitivity | λ | Photopic sensitivity |
|---|---|---|---|---|---|
| 380 | 0,000039 | 515 | 0,6082 | 650 | 0,107 |
| 381 | 4,28264E-05 | 516 | 0,6293456 | 651 | 0,1014762 |
| 382 | 4,69146E-05 | 517 | 0,6503068 | 652 | 0,09618864 |
| 383 | 5,15896E-05 | 518 | 0,6708752 | 653 | 0,09112296 |
| 384 | 5,71764E-05 | 519 | 0,6908424 | 654 | 0,08626485 |
| 385 | 0,000064 | 520 | 0,71 | 655 | 0,0816 |
| 386 | 7,23442E-05 | 521 | 0,7281852 | 656 | 0,07712064 |
| 387 | 8,22122E-05 | 522 | 0,7454636 | 657 | 0,07282552 |
| 388 | 9,35082E-05 | 523 | 0,7619694 | 658 | 0,06871008 |
| 389 | 0,000106136 | 524 | 0,7778368 | 659 | 0,06476976 |
| 390 | 0,00012 | 525 | 0,7932 | 660 | 0,061 |
| 391 | 0,000134984 | 526 | 0,8081104 | 661 | 0,05739621 |
| 392 | 0,000151492 | 527 | 0,8224962 | 662 | 0,05395504 |
| 393 | 0,000170208 | 528 | 0,8363068 | 663 | 0,05067376 |
| 394 | 0,000191816 | 529 | 0,8494916 | 664 | 0,04754965 |
| 395 | 0,000217 | 530 | 0,862 | 665 | 0,04458 |
| 396 | 0,000246907 | 531 | 0,8738108 | 666 | 0,04175872 |
| 397 | 0,00028124 | 532 | 0,8849624 | 667 | 0,03908496 |
| 398 | 0,00031852 | 533 | 0,8954936 | 668 | 0,03656384 |
| 399 | 0,000357267 | 534 | 0,9054432 | 669 | 0,03420048 |
| 400 | 0,000396 | 535 | 0,9148501 | 670 | 0,032 |
| 401 | 0,000433715 | 536 | 0,9237348 | 671 | 0,02996261 |
| 402 | 0,000473024 | 537 | 0,9320924 | 672 | 0,02807664 |
| 403 | 0,000517876 | 538 | 0,9399226 | 673 | 0,02632936 |
| 404 | 0,000572219 | 539 | 0,9472252 | 674 | 0,02470805 |
| 405 | 0,00064 | 540 | 0,954 | 675 | 0,0232 |
| 406 | 0,00072456 | 541 | 0,9602561 | 676 | 0,02180077 |
| 407 | 0,0008255 | 542 | 0,9660074 | 677 | 0,02050112 |
| 408 | 0,00094116 | 543 | 0,9712606 | 678 | 0,01928108 |
| 409 | 0,00106988 | 544 | 0,9760225 | 679 | 0,01812069 |
| 410 | 0,00121 | 545 | 0,9803 | 680 | 0,017 |
| 411 | 0,001362091 | 546 | 0,9840924 | 681 | 0,01590379 |
| 412 | 0,001530752 | 547 | 0,9874182 | 682 | 0,01483718 |
| 413 | 0,001720368 | 548 | 0,9903128 | 683 | 0,01381068 |
| 414 | 0,001935323 | 549 | 0,9928116 | 684 | 0,01283478 |
| 415 | 0,00218 | 550 | 0,9949501 | 685 | 0,01192 |
| 416 | 0,0024548 | 551 | 0,9967108 | 686 | 0,01106831 |
| 417 | 0,002764 | 552 | 0,9980983 | 687 | 0,01027339 |
| 418 | 0,0031178 | 553 | 0,999112 | 688 | 0,009533311 |
| 419 | 0,0035264 | 554 | 0,9997482 | 689 | 0,008846157 |
| 420 | 0,004 | 555 | 1 | 690 | 0,00821 |
| 421 | 0,00454624 | 556 | 0,9998567 | 691 | 0,007623781 |
| 422 | 0,00515932 | 557 | 0,9993046 | 692 | 0,007085424 |
| 423 | 0,00582928 | 558 | 0,9983255 | 693 | 0,006591476 |
| 424 | 0,00654616 | 559 | 0,9968987 | 694 | 0,006138485 |
| 425 | 0,0073 | 560 | 0,995 | 695 | 0,005723 |
| 426 | 0,008086507 | 561 | 0,9926005 | 696 | 0,005343059 |
| 427 | 0,00890872 | 562 | 0,9897426 | 697 | 0,004995796 |
| 428 | 0,00976768 | 563 | 0,9864444 | 698 | 0,004676404 |
| 429 | 0,01066443 | 564 | 0,9827241 | 699 | 0,004380075 |
| 430 | 0,0116 | 565 | 0,9786 | 700 | 0,004102 |
| 431 | 0,01257317 | 566 | 0,9740837 | 701 | 0,003838453 |
| 432 | 0,01358272 | 567 | 0,9691712 | 702 | 0,003589099 |
| 433 | 0,01462968 | 568 | 0,9638568 | 703 | 0,003354219 |
| 434 | 0,01571509 | 569 | 0,9581349 | 704 | 0,003134093 |
| 435 | 0,01684 | 570 | 0,952 | 705 | 0,002929 |
| 436 | 0,01800736 | 571 | 0,9454504 | 706 | 0,002738139 |
| 437 | 0,01921448 | 572 | 0,9384992 | 707 | 0,002559876 |
| 438 | 0,02045392 | 573 | 0,9311628 | 708 | 0,002393244 |
| 439 | 0,02171824 | 574 | 0,9234576 | 709 | 0,002237275 |
| 440 | 0,023 | 575 | 0,9154 | 710 | 0,002091 |
| 441 | 0,02429461 | 576 | 0,9070064 | 711 | 0,001953587 |
| 442 | 0,02561024 | 577 | 0,8982772 | 712 | 0,00182458 |
| 443 | 0,02695857 | 578 | 0,8892048 | 713 | 0,00170358 |
| 444 | 0,02835125 | 579 | 0,8797816 | 714 | 0,001590187 |
| 445 | 0,0298 | 580 | 0,87 | 715 | 0,001484 |
| 446 | 0,03131083 | 581 | 0,8598613 | 716 | 0,001384496 |
| 447 | 0,03288368 | 582 | 0,849392 | 717 | 0,001291268 |
| 448 | 0,03452112 | 583 | 0,838622 | 718 | 0,001204092 |
| 449 | 0,03622571 | 584 | 0,8275813 | 719 | 0,001122744 |
| 450 | 0,038 | 585 | 0,8163 | 720 | 0,001047 |
| 451 | 0,03984667 | 586 | 0,8047947 | 721 | 0,00097659 |
| 452 | 0,041768 | 587 | 0,793082 | 722 | 0,000911109 |
| 453 | 0,043766 | 588 | 0,781192 | 723 | 0,000850133 |
| 454 | 0,04584267 | 589 | 0,7691547 | 724 | 0,000793238 |
| 455 | 0,048 | 590 | 0,757 | 725 | 0,00074 |
| 456 | 0,05024368 | 591 | 0,7447541 | 726 | 0,000690083 |
| 457 | 0,05257304 | 592 | 0,7324224 | 727 | 0,00064331 |
| 458 | 0,05498056 | 593 | 0,7200036 | 728 | 0,000599496 |
| 459 | 0,05745872 | 594 | 0,7074965 | 729 | 0,000558455 |
| 460 | 0,06 | 595 | 0,6949 | 730 | 0,00052 |
| 461 | 0,06260197 | 596 | 0,6822192 | 731 | 0,000483914 |
| 462 | 0,06527752 | 597 | 0,6694716 | 732 | 0,000450053 |
| 463 | 0,06804208 | 598 | 0,6566744 | 733 | 0,000418345 |
| 464 | 0,07091109 | 599 | 0,6438448 | 734 | 0,000388718 |
| 465 | 0,0739 | 600 | 0,631 | 735 | 0,0003611 |
| 466 | 0,077016 | 601 | 0,6181555 | 736 | 0,000335384 |
| 467 | 0,0802664 | 602 | 0,6053144 | 737 | 0,00031144 |
| 468 | 0,0836668 | 603 | 0,5924756 | 738 | 0,000289166 |
| 469 | 0,0872328 | 604 | 0,5796379 | 739 | 0,000268454 |
| 470 | 0,09098 | 605 | 0,5668 | 740 | 0,0002492 |
| 471 | 0,09491755 | 606 | 0,5539611 | 741 | 0,000231302 |
| 472 | 0,09904584 | 607 | 0,5411372 | 742 | 0,000214686 |
| 473 | 0,1033674 | 608 | 0,5283528 | 743 | 0,000199288 |
| 474 | 0,1078846 | 609 | 0,5156323 | 744 | 0,000185048 |
| 475 | 0,1126 | 610 | 0,503 | 745 | 0,0001719 |
| 476 | 0,117532 | 611 | 0,4904688 | 746 | 0,000159778 |
| 477 | 0,1226744 | 612 | 0,4780304 | 747 | 0,000148604 |
| 478 | 0,1279928 | 613 | 0,4656776 | 748 | 0,000138302 |
| 479 | 0,1334528 | 614 | 0,4534032 | 749 | 0,000128793 |
| 480 | 0,13902 | 615 | 0,4412 | 750 | 0,00012 |
| 481 | 0,1446764 | 616 | 0,42908 | 751 | 0,00011186 |
| 482 | 0,1504693 | 617 | 0,417036 | 752 | 0,000104322 |
| 483 | 0,1564619 | 618 | 0,405032 | 753 | 9,73356E-05 |
| 484 | 0,1627177 | 619 | 0,393032 | 754 | 9,08459E-05 |
| 485 | 0,1693 | 620 | 0,381 | 755 | 0,0000848 |
| 486 | 0,1762431 | 621 | 0,3689184 | 756 | 7,91467E-05 |
| 487 | 0,1835581 | 622 | 0,3568272 | 757 | 0,000073858 |
| 488 | 0,1912735 | 623 | 0,3447768 | 758 | 0,000068916 |
| 489 | 0,199418 | 624 | 0,3328176 | 759 | 6,43027E-05 |
| 490 | 0,20802 | 625 | 0,321 | 760 | 0,00006 |
| 491 | 0,2171199 | 626 | 0,3093381 | 761 | 5,59819E-05 |
| 492 | 0,2267345 | 627 | 0,2978504 | 762 | 5,22256E-05 |
| 493 | 0,2368571 | 628 | 0,2865936 | 763 | 4,87184E-05 |
| 494 | 0,2474812 | 629 | 0,2756245 | 764 | 4,54475E-05 |
| 495 | 0,2586 | 630 | 0,265 | 765 | 0,0000424 |
| 496 | 0,2701849 | 631 | 0,2547632 | 766 | 3,9561E-05 |
| 497 | 0,2822939 | 632 | 0,2448896 | 767 | 3,69151E-05 |
| 498 | 0,2950505 | 633 | 0,2353344 | 768 | 3,44487E-05 |
| 499 | 0,308578 | 634 | 0,2260528 | 769 | 3,21482E-05 |
| 500 | 0,323 | 635 | 0,217 | 770 | 0,00003 |
| 501 | 0,3384021 | 636 | 0,2081616 | 771 | 2,79913E-05 |
| 502 | 0,3546858 | 637 | 0,1995488 | 772 | 2,61136E-05 |
| 503 | 0,3716986 | 638 | 0,1911552 | 773 | 2,43602E-05 |
| 504 | 0,3892875 | 639 | 0,1829744 | 774 | 2,27246E-05 |
| 505 | 0,4073 | 640 | 0,175 | 775 | 0,0000212 |
| 506 | 0,4256299 | 641 | 0,1672235 | 776 | 1,97789E-05 |
| 507 | 0,4443096 | 642 | 0,1596464 | 777 | 1,84529E-05 |
| 508 | 0,4633944 | 643 | 0,1522776 | 778 | 1,72169E-05 |
| 509 | 0,4829395 | 644 | 0,1451259 | 779 | 1,60646E-05 |
| 510 | 0,503 | 645 | 0,1382 | 780 | 0,00001499 |
| 511 | 0,5235693 | 646 | 0,1315003 | | |
| 512 | 0,544512 | 647 | 0,1250248 | | |
| 513 | 0,56569 | 648 | 0,1187792 | | |
| 514 | 0,5869653 | 649 | 0,1127691 | | |
| 515 | 0,6082 | | | | |

The activation spectrum for the cytochrome c oxidase chromophore appears to differ for DNA synthesis and RNA synthesis. The activation spectra are known from the art and are herein provided in Table 3:

TABLE 3

activation spectra (s_{cyt}(λ)) for the cytochrome c oxidase chromophore for DNA and RNA synthesis, respectively

| λ (nm) | s_{cyt}(λ) DNA | s_{cyt}(λ) RNA | λ (nm) | s_{cyt}(λ) DNA | s_{cyt}(λ) RNA | λ (nm) | s_{cyt}(λ) DNA | s_{cyt}(A) RNA |
|---|---|---|---|---|---|---|---|---|
| 550 | 0.026 | 0.038 | 670 | 0.443 | 0.238 | 790 | 0.263 | 0.397 |
| 551 | 0.026 | 0.039 | 671 | 0.445 | 0.255 | 791 | 0.266 | 0.371 |
| 552 | 0.027 | 0.040 | 672 | 0.440 | 0.275 | 792 | 0.270 | 0.347 |
| 553 | 0.028 | 0.041 | 673 | 0.430 | 0.299 | 793 | 0.276 | 0.325 |
| 554 | 0.028 | 0.042 | 674 | 0.414 | 0.326 | 794 | 0.284 | 0.305 |
| 555 | 0.029 | 0.043 | 675 | 0.394 | 0.357 | 795 | 0.294 | 0.287 |
| 556 | 0.030 | 0.044 | 676 | 0.372 | 0.392 | 796 | 0.305 | 0.271 |
| 557 | 0.031 | 0.045 | 677 | 0.349 | 0.431 | 797 | 0.317 | 0.256 |
| 558 | 0.031 | 0.047 | 678 | 0.325 | 0.471 | 798 | 0.331 | 0.242 |
| 559 | 0.032 | 0.048 | 679 | 0.302 | 0.512 | 799 | 0.347 | 0.229 |
| 560 | 0.033 | 0.049 | 680 | 0.280 | 0.550 | 800 | 0.363 | 0.218 |
| 561 | 0.034 | 0.051 | 681 | 0.259 | 0.581 | 801 | 0.381 | 0.208 |
| 562 | 0.035 | 0.052 | 682 | 0.240 | 0.602 | 802 | 0.399 | 0.198 |
| 563 | 0.036 | 0.054 | 683 | 0.222 | 0.609 | 803 | 0.418 | 0.190 |
| 564 | 0.037 | 0.056 | 684 | 0.207 | 0.601 | 804 | 0.436 | 0.182 |
| 565 | 0.038 | 0.057 | 685 | 0.192 | 0.579 | 805 | 0.453 | 0.175 |
| 566 | 0.039 | 0.059 | 686 | 0.179 | 0.546 | 806 | 0.468 | 0.169 |
| 567 | 0.040 | 0.061 | 687 | 0.168 | 0.507 | 807 | 0.480 | 0.164 |
| 568 | 0.041 | 0.063 | 688 | 0.157 | 0.465 | 808 | 0.489 | 0.160 |
| 569 | 0.043 | 0.065 | 689 | 0.148 | 0.423 | 809 | 0.494 | 0.156 |
| 570 | 0.044 | 0.068 | 690 | 0.140 | 0.384 | 810 | 0.495 | 0.153 |
| 571 | 0.045 | 0.070 | 691 | 0.132 | 0.347 | 811 | 0.491 | 0.151 |
| 572 | 0.047 | 0.073 | 692 | 0.125 | 0.315 | 812 | 0.483 | 0.151 |
| 573 | 0.048 | 0.075 | 693 | 0.119 | 0.286 | 813 | 0.471 | 0.151 |
| 574 | 0.050 | 0.078 | 694 | 0.114 | 0.261 | 814 | 0.455 | 0.152 |
| 575 | 0.052 | 0.081 | 695 | 0.109 | 0.239 | 815 | 0.437 | 0.155 |
| 576 | 0.053 | 0.084 | 696 | 0.104 | 0.220 | 816 | 0.416 | 0.159 |
| 577 | 0.055 | 0.088 | 697 | 0.100 | 0.204 | 817 | 0.395 | 0.166 |
| 578 | 0.057 | 0.091 | 698 | 0.097 | 0.190 | 818 | 0.373 | 0.176 |
| 579 | 0.059 | 0.095 | 699 | 0.093 | 0.177 | 819 | 0.351 | 0.189 |
| 580 | 0.062 | 0.099 | 700 | 0.090 | 0.166 | 820 | 0.329 | 0.207 |
| 581 | 0.064 | 0.103 | 701 | 0.088 | 0.157 | 821 | 0.308 | 0.232 |
| 582 | 0.066 | 0.108 | 702 | 0.085 | 0.149 | 822 | 0.289 | 0.265 |
| 583 | 0.069 | 0.113 | 703 | 0.083 | 0.142 | 823 | 0.270 | 0.308 |
| 584 | 0.072 | 0.118 | 704 | 0.081 | 0.136 | 824 | 0.252 | 0.364 |
| 585 | 0.075 | 0.124 | 705 | 0.079 | 0.130 | 825 | 0.236 | 0.432 |
| 586 | 0.078 | 0.130 | 706 | 0.078 | 0.125 | 826 | 0.221 | 0.505 |
| 587 | 0.081 | 0.136 | 707 | 0.076 | 0.121 | 827 | 0.207 | 0.565 |
| 588 | 0.085 | 0.143 | 708 | 0.075 | 0.118 | 828 | 0.194 | 0.587 |
| 589 | 0.089 | 0.150 | 709 | 0.074 | 0.115 | 829 | 0.182 | 0.561 |
| 590 | 0.093 | 0.158 | 710 | 0.073 | 0.112 | 830 | 0.171 | 0.497 |
| 591 | 0.097 | 0.167 | 711 | 0.073 | 0.110 | 831 | 0.160 | 0.420 |
| 592 | 0.102 | 0.176 | 712 | 0.072 | 0.108 | 832 | 0.151 | 0.348 |
| 593 | 0.106 | 0.186 | 713 | 0.072 | 0.107 | 833 | 0.142 | 0.288 |
| 594 | 0.112 | 0.196 | 714 | 0.072 | 0.105 | 834 | 0.134 | 0.240 |
| 595 | 0.117 | 0.208 | 715 | 0.071 | 0.104 | 835 | 0.127 | 0.203 |
| 596 | 0.124 | 0.220 | 716 | 0.071 | 0.104 | 836 | 0.120 | 0.175 |
| 597 | 0.130 | 0.234 | 717 | 0.071 | 0.103 | 837 | 0.114 | 0.152 |
| 598 | 0.137 | 0.248 | 718 | 0.072 | 0.103 | 838 | 0.108 | 0.134 |
| 599 | 0.145 | 0.264 | 719 | 0.072 | 0.103 | 839 | 0.103 | 0.120 |
| 600 | 0.153 | 0.281 | 720 | 0.073 | 0.104 | 840 | 0.098 | 0.108 |
| 601 | 0.162 | 0.299 | 721 | 0.073 | 0.104 | 841 | 0.093 | 0.098 |
| 602 | 0.171 | 0.319 | 722 | 0.074 | 0.105 | 842 | 0.089 | 0.090 |
| 603 | 0.182 | 0.340 | 723 | 0.075 | 0.106 | 843 | 0.085 | 0.083 |
| 604 | 0.193 | 0.363 | 724 | 0.076 | 0.107 | 844 | 0.081 | 0.078 |
| 605 | 0.204 | 0.388 | 725 | 0.077 | 0.109 | 845 | 0.077 | 0.072 |
| 606 | 0.217 | 0.414 | 726 | 0.079 | 0.110 | 846 | 0.074 | 0.068 |
| 607 | 0.231 | 0.441 | 727 | 0.080 | 0.112 | 847 | 0.071 | 0.064 |
| 608 | 0.246 | 0.470 | 728 | 0.082 | 0.114 | 848 | 0.068 | 0.061 |
| 609 | 0.261 | 0.499 | 729 | 0.084 | 0.117 | 849 | 0.065 | 0.058 |
| 610 | 0.278 | 0.530 | 730 | 0.086 | 0.119 | 850 | 0.063 | 0.055 |
| 611 | 0.296 | 0.560 | 731 | 0.089 | 0.122 | 851 | 0.061 | 0.052 |
| 612 | 0.314 | 0.591 | 732 | 0.092 | 0.126 | 852 | 0.058 | 0.050 |
| 613 | 0.334 | 0.620 | 733 | 0.095 | 0.129 | 853 | 0.056 | 0.048 |
| 614 | 0.354 | 0.646 | 734 | 0.098 | 0.133 | 854 | 0.054 | 0.046 |
| 615 | 0.374 | 0.670 | 735 | 0.102 | 0.137 | 855 | 0.052 | 0.044 |
| 616 | 0.394 | 0.690 | 736 | 0.106 | 0.142 | 856 | 0.050 | 0.043 |
| 617 | 0.414 | 0.705 | 737 | 0.110 | 0.147 | 857 | 0.049 | 0.041 |
| 618 | 0.433 | 0.715 | 738 | 0.115 | 0.153 | 858 | 0.047 | 0.040 |
| 619 | 0.451 | 0.718 | 739 | 0.121 | 0.159 | 859 | 0.046 | 0.039 |
| 620 | 0.467 | 0.716 | 740 | 0.127 | 0.165 | 860 | 0.044 | 0.037 |
| 621 | 0.480 | 0.707 | 741 | 0.134 | 0.172 | 861 | 0.043 | 0.036 |
| 622 | 0.490 | 0.693 | 742 | 0.141 | 0.180 | 862 | 0.041 | 0.035 |
| 623 | 0.497 | 0.673 | 743 | 0.150 | 0.188 | 863 | 0.040 | 0.034 |
| 624 | 0.500 | 0.650 | 744 | 0.159 | 0.197 | 864 | 0.039 | 0.033 |
| 625 | 0.499 | 0.624 | 745 | 0.170 | 0.207 | 865 | 0.038 | 0.032 |
| 626 | 0.494 | 0.596 | 746 | 0.181 | 0.218 | 866 | 0.037 | 0.031 |
| 627 | 0.486 | 0.567 | 747 | 0.195 | 0.230 | 867 | 0.036 | 0.031 |
| 628 | 0.474 | 0.537 | 748 | 0.210 | 0.243 | 868 | 0.035 | 0.030 |
| 629 | 0.460 | 0.507 | 749 | 0.227 | 0.257 | 869 | 0.034 | 0.029 |
| 630 | 0.444 | 0.479 | 750 | 0.246 | 0.272 | 870 | 0.033 | 0.028 |
| 631 | 0.427 | 0.451 | 751 | 0.269 | 0.289 | 871 | 0.032 | 0.028 |
| 632 | 0.409 | 0.424 | 752 | 0.294 | 0.307 | 872 | 0.031 | 0.027 |
| 633 | 0.391 | 0.399 | 753 | 0.323 | 0.327 | 873 | 0.030 | 0.026 |
| 634 | 0.373 | 0.376 | 754 | 0.357 | 0.349 | 874 | 0.030 | 0.026 |
| 635 | 0.355 | 0.354 | 755 | 0.395 | 0.373 | 875 | 0.029 | 0.025 |
| 636 | 0.338 | 0.334 | 756 | 0.439 | 0.399 | 876 | 0.028 | 0.025 |
| 637 | 0.322 | 0.315 | 757 | 0.489 | 0.427 | 877 | 0.027 | 0.024 |
| 638 | 0.307 | 0.298 | 758 | 0.546 | 0.458 | 878 | 0.027 | 0.024 |
| 639 | 0.293 | 0.282 | 759 | 0.609 | 0.492 | 879 | 0.026 | 0.023 |
| 640 | 0.280 | 0.268 | 760 | 0.677 | 0.528 | 880 | 0.026 | 0.023 |
| 641 | 0.268 | 0.255 | 761 | 0.750 | 0.567 | 881 | 0.025 | 0.022 |
| 642 | 0.258 | 0.243 | 762 | 0.823 | 0.608 | 882 | 0.024 | 0.022 |
| 643 | 0.249 | 0.231 | 763 | 0.891 | 0.652 | 883 | 0.024 | 0.021 |
| 644 | 0.241 | 0.221 | 764 | 0.947 | 0.698 | 884 | 0.023 | 0.021 |
| 645 | 0.234 | 0.212 | 765 | 0.986 | 0.745 | 885 | 0.023 | 0.021 |
| 646 | 0.228 | 0.204 | 766 | 1.000 | 0.792 | 886 | 0.022 | 0.020 |
| 647 | 0.223 | 0.197 | 767 | 0.989 | 0.838 | 887 | 0.022 | 0.020 |
| 648 | 0.220 | 0.190 | 768 | 0.954 | 0.882 | 888 | 0.021 | 0.019 |
| 649 | 0.217 | 0.184 | 769 | 0.900 | 0.921 | 889 | 0.021 | 0.019 |
| 650 | 0.216 | 0.179 | 770 | 0.835 | 0.954 | 890 | 0.021 | 0.019 |
| 651 | 0.216 | 0.175 | 771 | 0.766 | 0.979 | 891 | 0.020 | 0.018 |
| 652 | 0.217 | 0.171 | 772 | 0.696 | 0.995 | 892 | 0.020 | 0.018 |
| 653 | 0.219 | 0.168 | 773 | 0.631 | 1.000 | 893 | 0.019 | 0.018 |
| 654 | 0.223 | 0.165 | 774 | 0.572 | 0.995 | 894 | 0.019 | 0.018 |
| 655 | 0.228 | 0.163 | 775 | 0.519 | 0.979 | 895 | 0.019 | 0.017 |
| 656 | 0.235 | 0.162 | 776 | 0.472 | 0.954 | 896 | 0.018 | 0.017 |
| 657 | 0.243 | 0.161 | 777 | 0.433 | 0.920 | 897 | 0.018 | 0.017 |
| 658 | 0.252 | 0.161 | 778 | 0.398 | 0.881 | 898 | 0.018 | 0.016 |
| 659 | 0.264 | 0.162 | 779 | 0.369 | 0.837 | 899 | 0.017 | 0.016 |
| 660 | 0.277 | 0.163 | 780 | 0.345 | 0.791 | 900 | 0.017 | 0.016 |
| 661 | 0.292 | 0.165 | 781 | 0.325 | 0.744 | | | |
| 662 | 0.309 | 0.168 | 782 | 0.308 | 0.697 | | | |
| 663 | 0.328 | 0.172 | 783 | 0.294 | 0.651 | | | |
| 664 | 0.348 | 0.177 | 784 | 0.283 | 0.607 | | | |
| 665 | 0.368 | 0.183 | 785 | 0.275 | 0.565 | | | |
| 666 | 0.388 | 0.191 | 786 | 0.268 | 0.526 | | | |
| 667 | 0.407 | 0.200 | 787 | 0.264 | 0.490 | | | |
| 668 | 0.423 | 0.210 | 788 | 0.262 | 0.456 | | | |
| 669 | 0.436 | 0.223 | 789 | 0.261 | 0.425 | | | |

The spectral sensitivity data of cytochrome C oxidase activation as listed in Table 3 are normalized to 1. The most pronounced and efficient activation seems to be in the wavelengths between 550-900 nm. For wavelengths which activate cytochrome C oxidase, i.e. within a range from 550 nm to 900 nm, a kind of efficacy can be defined, semi analogous to the photopic luminous efficacy for visible light but now in relation to light for cytochrome c oxidase activation. In this specification, such efficacy is referred to as cytochrome C oxidase efficacy and is defined as the spectral power in the spectral range of 550-900 nm weighted with the cytochrome c oxidase activation curves for respectively DNA synthesis and RNA synthesis, respectively, relative to the spectral power in the spectral range of 380-780 nm weighted with the luminosity function of the human eye.

The cytochrome c oxidase efficacy also appears to differ for DNA and RNA synthesis, which is the reason why the cytochrome C oxidase efficacy related conditions are defined for DNA and RNA synthesis respectively.

In the embodiment of the lighting device 1 shown in FIG. 1, the lighting device 1 comprises one processor 5. In an alternative embodiment, the lighting device 1 comprises multiple processors. The processor 5 of the lighting device 1 may be a general-purpose processor or an application-specific processor. The receiver 3 and the transmitter 4 may use one or more wireless communication technologies. e.g. Zigbee, for communicating with the bridge 21. In an alternative embodiment, multiple receivers and/or multiple transmitters are used instead of a single receiver and a single transmitter.

In the embodiment shown in FIG. 1, a separate receiver and a separate transmitter are used. In an alternative embodiment, the receiver 3 and the transmitter 4 are combined into a transceiver. The lighting device 1 may comprise other hardware components typical for a connected lighting device such as a power connector and a memory. In an alternative embodiment, the lighting device 1 is not a connected lighting device. The invention may be implemented using a computer program running on one or more processors.

In the embodiment of FIG. 1, the system of the invention is a lighting device. In an alternative embodiment, the system of the invention is a different device, e.g. a mobile device or a controller. In the embodiments of FIG. 1, the system of the invention comprises a single device. In an alternative embodiment, the system of the invention comprises a plurality of devices.

FIG. 2 shows a second embodiment of the system for controlling one or more light sources to render light comprising a light component having wavelengths in the range 550 to 900 nm: a mobile device 41. A lighting device 51 is capable of rendering white light and comprises visible-light LED 11 of FIG. 1. A lighting device 52 is capable of rendering UV-B light and comprises UV-B LED 12 of FIG. 1. Lighting devices 51 and 52 are typically co-located. A lighting system 59 comprises the lighting devices 51-52 and the bridge 21.

The mobile device 41 comprises a receiver 43, a transmitter 44, a processor 45, memory 47, and a display 49. The processor 45 is configured to determine whether a person has been and/or will be irradiated with an amount of UV radiation exceeding a threshold and control, via the transmitter 45, in dependence on the determination, the lighting device 51 (and thereby visible-light LED 11) to render light comprising a light component having wavelengths in the range 550 to 900 nm. The light component preferably comprises wavelengths in the range 600 to 850 nm, e.g. in the range 605 to 635 nm, in the range 660 to 690 nm, in the range 755 to 790 nm and/or in the range 800 to 835 nm.

A spectral power distribution of the light is chosen such that the light has a cytochrome C oxidase efficacy complying with one or more of the following conditions:
(i) the cytochrome C oxidase efficacy for DNA synthesis is at least (6.2*vʹ-2.48) mW/lm if the light’s vʹ is lower than 0.539 or is at least 0.85 mW/lm if the light’s vʹ is equal to or higher than 0.539, and
(ii) the cytochrome C oxidase efficacy for RNA synthesis is at least (7.5*vʹ-2.975) mW/lm if the light’s vʹ is lower than 0.539 or is at least 1.05 mW/lm if the light’s vʹ is equal to or higher than 0.539,
wherein the cytochrome C oxidase efficacy is defined as:

$$\begin{matrix}C y t o c h r o m e C o x i d a s e E f f i c a c y o f r a d i a t i o n \left(W / L m\right) = \\ \frac{{\int }_{550}^{900} {Φ}_{e , λ} {s}_{c y t} \left(λ\right) d λ}{683 {\int }_{380}^{780} {Φ}_{e , λ} \left(λ\right) V \left(λ\right) d λ}\end{matrix}$$

wherein:
Φ_{e,λ}(λ) = is the spectral power distribution of the light
s_{cyt}(λ) = is the spectral sensitivity of cytochrome C oxidase activation for either DNA synthesis or RNA synthesis;
V(λ) = is the photopic luminosity function; and
vʹ is a color coordinate of the light in the CIE 1976 Uniform Chromaticity Scale diagram.

In the embodiment of FIG. 2, the processor 45 is configured to determine an amount of UV radiation received by a light sensor comprised in a personal device 61 and determine whether the person has been irradiated with an amount of UV radiation exceeding the threshold based on the determined amount of UV radiation received by this light sensor. The personal device 61 transmits information indicating the amount of received UV radiation to the mobile device 41. The personal device 61 may be a smart watch, for example.

The amount (both time and intensity) of the UV(-B) irradiation to which the user has been exposed is recorded by the mobile device 41 and/or personal device 61. Optionally, the exposure to other light spectra is also recorded. The cytochrome C stimulating contribution of the rendered visible light may be increased (by changing the spectral power distribution of the light) based on the recorded amount of UV(-B) irradiation.

In the embodiment of FIG. 2, the processor 45 is configured to control, via the transmitter 44, the lighting device 52 (and thereby UV-B LED 12) to render the light (which is jointly rendered by lighting devices 51-52) such that the light includes at least part of the UV radiation. A user of the mobile device 45 may be able to use an app on the mobile device 41 to start and stop UV irradiation, for example.

In an alternative embodiment, the mobile device 41 does not need to determine an amount of UV radiation received by a light sensor, but is able to determine whether the person has been irradiated with an amount of UV radiation exceeding the threshold based on the control signals that it has transmitted to the lighting device 52 by a transmitter 44 or based on a schedule that has resulted in and/or will result in the transmission of control signals to the lighting device 52 by the transmitter 44. Determining the amount of UV radiation received by a light sensor is especially beneficial when no UV radiation information is received from the lighting device 52 or the system that controls the lighting device 52 and also allows the UV radiation received from the sun to be determined.

The user may also be able to change a color and/or intensity of the visible light, e.g. using the (touch screen) display 49. The light rendered by the lighting device 51 may comprise further light components which make the light look white. The term white light relates to light having a correlated color temperature (CCT) between about 2000 K and 20000 K and within about 10 to 15 SDCM (standard deviation of color matching) from the BBL (black body locus).

In the embodiment of FIG. 2, the lighting devices 51-52 each comprise only one LED. In an alternative embodiment, one or more of the lighting devices 51-52 comprise multiple LEDs, typically of the same kind (visible-light or UV-B), as also described in relation to the LED module 9 of FIG. 1.

In the embodiment of FIG. 2, the mobile device 41 and the lighting devices 51-52 communicate via the bridge 21. In an alternative embodiment, multiple of the mobile device 41 and the lighting devices 51-52 can alternatively or additionally communicate directly, e.g. using Bluetooth technology.

In the embodiment of the mobile device 41 shown in FIG. 2, the mobile device 41 comprises one processor 45. In an alternative embodiment, the mobile device 1 comprises multiple processors. The processor 45 of the mobile device 41 may be a general-purpose processor, e.g. from ARM or Qualcomm or an application-specific processor. The processor 45 of the mobile device 41 may run an Android or iOS operating system for example. The display 49 may comprise an LCD or OLED display panel, for example. The display 49 may be a touch screen display, for example. The memory 47 may comprise one or more memory units. The memory 47 may comprise solid state memory, for example.

The receiver 43 and the transmitter 44 may use one or more wireless communication technologies, e.g. Wi-Fi (IEEE 802.11) for communicating with the wireless LAN access point 23, for example. In an alternative embodiment, multiple receivers and/or multiple transmitters are used instead of a single receiver and a single transmitter. In the embodiment shown in FIG. 2, a separate receiver and a separate transmitter are used. In an alternative embodiment, the receiver 43 and the transmitter 44 are combined into a transceiver. The mobile device 41 may comprise other hardware components typical for a mobile device such as a battery and a power connector. The invention may be implemented using a computer program running on one or more processors.

FIG. 3 shows a third embodiment of the system for controlling one or more light sources to render light comprising a component having wavelengths in the range 550 to 900 nm: a controller 81, e.g. a bridge or a gateway. A lighting system 99 comprises the lighting devices 51-52 and the controller 81.

The controller 81 comprises a receiver 83, a transmitter 84, a processor 85, and memory 87. The processor 85 is configured to determine whether a person has been and/or will be irradiated with an amount of UV radiation exceeding a threshold and control, via the transmitter 84, in dependence on the determination, the lighting device 51 (and thereby visible-light LED 11) to render light comprising a light component having wavelengths in the range 550 to 900 nm. The light component preferably comprises wavelengths in the range 600 to 850 nm, e.g. in the range 605 to 635 nm, in the range 660 to 690 nm, in the range 755 to 790 nm and/or in the range 800 to 835 nm.

A spectral power distribution of the light is chosen such that the light has a cytochrome C oxidase efficacy complying with one or more of the following conditions:
(i) the cytochrome C oxidase efficacy for DNA synthesis is at least (6.2*vʹ-2.48) mW/lm if the light’s vʹ is lower than 0.539 or is at least 0.85 mW/lm if the light’s vʹ is equal to or higher than 0.539, and
(ii) the cytochrome C oxidase efficacy for RNA synthesis is at least (7.5*vʹ-2.975) mW/lm if the light’s vʹ is lower than 0.539 or is at least 1.05 mW/lm if the light’s vʹ is equal to or higher than 0.539,
wherein the cytochrome C oxidase efficacy is defined as:

$$\begin{matrix}C y t o c h r o m e C o x i d a s e E f f i c a c y o f r a d i a t i o n \left(W / L m\right) = \\ \frac{{\int }_{550}^{900} {Φ}_{\text{e,} λ} \left(λ\right) {s}_{c y t} \left(λ\right) d λ}{683 {\int }_{380}^{780} {Φ}_{\text{e,} λ} \left(λ\right) V \left(λ\right) d λ}\end{matrix}$$

wherein:
Φ_{e,λ}(λ) = is the spectral power distribution of the light
s_{cyt}(λ) = is the spectral sensitivity of cytochrome C oxidase activation for either DNA synthesis or RNA synthesis;
V(λ) = is the photopic luminosity function; and
vʹ is a color coordinate of the light in the CIE 1976 Uniform Chromaticity Scale diagram.

In the embodiment of FIG. 3, the processor 85 is configured to receive UV radiation information from lighting device 52 and determine whether the person has been irradiated with an amount of UV radiation exceeding the threshold based on this radiation information. This radiation information indicates the amount of generated UV radiation, possibly associated with identifiers of users who (may) have been exposed to the UV radiation.

In the embodiment of the controller 81 shown in FIG. 3, the controller 81 comprises one processor 85. In an alternative embodiment, the controller 81 comprises multiple processors. The processor 85 of the controller 81 may be a general-purpose processor, e.g. ARM-based, or an application-specific processor. The processor 85 of the controller 81 may run a Unix-based operating system for example. The memory 87 may comprise one or more memory units. The memory 87 may comprise one or more hard disks and/or solid-state memory, for example.

The receiver 83 and the transmitter 84 may use one or more wired or wireless communication technologies such as Zigbee to communicate with the lighting devices 51 and 52 and Ethernet to communicate with the wireless LAN access point 23, for example. In an alternative embodiment, multiple receivers and/or multiple transmitters are used instead of a single receiver and a single transmitter. In the embodiment shown in FIG. 3, a separate receiver and a separate transmitter are used. In an alternative embodiment, the receiver 83 and the transmitter 84 are combined into a transceiver. The controller 81 may comprise other hardware components typical for a controller such as a power connector. The invention may be implemented using a computer program running on one or more processors.

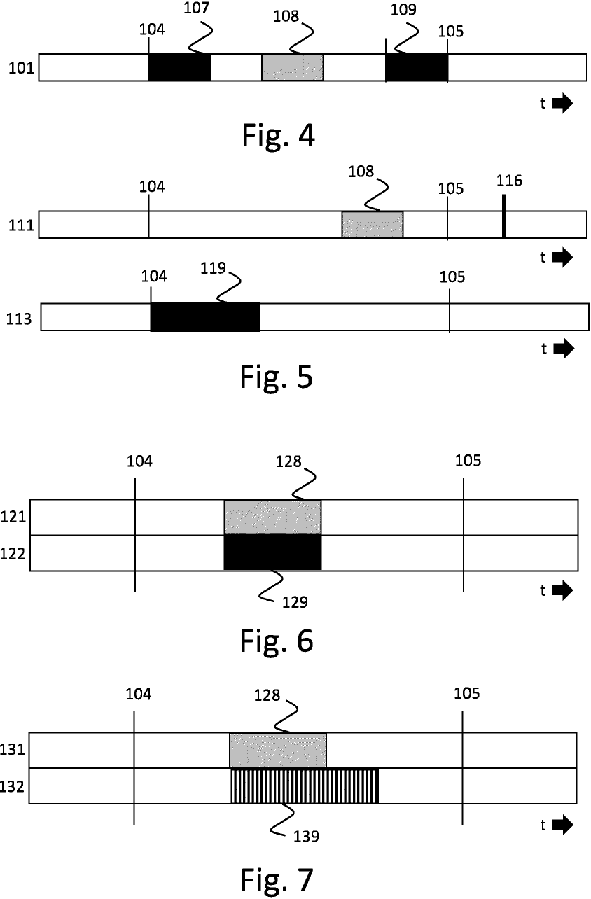

FIGS. 4 to 7 show examples of UV radiation and visible light being rendered over time. The visible light comprises a light component having wavelengths in the range 550 to 900 nm. This light component preferably comprises wavelengths in the range 600 to 850 nm, e.g. in the range 605 to 635 nm, in the range 660 to 690 nm, in the range 755 to 790 nm and/or in the range 800 to 835 nm. The visible light may look red or white, for example. For instance, the visible light may be rendered by deep red/NIR LEDs.

A daylight period starts at time 104 and ends at time 105. FIG. 4 shows that on day 101, the visible light is rendered in periods 107 and 109 and the UV radiation is rendered in period 108. It is possible to render the visible light before the UV radiation if it is known in advance by the system controlling the visible light when UV radiation is going to be rendered. The visible light may have circadian profile or an anti-circadian profile. The relative power in the spectral power distribution may be equal to natural light, for example.

As shown in FIG. 5, on day 111, UV radiation is rendered in period 108 by a first system. A second system is informed of this at time 116 and renders the visible light in period 119 of the next day, i.e. day 113. Alternatively, the second system might render the visible light shortly after being informed of the rendered UV radiation, i.e. shortly after time 116.

In the examples of FIGS. 6 and 7, the visible light is rendered at the same time the UV radiation is rendered, e.g. by the same system. In the example of FIG. 6, visible light is rendered continuously in period 128. In the example of FIG. 7, visible light is rendered in period 139 in a pulsating manner, e.g. every minute, at 1 Hz or at 0.1 Hz. In the example of FIG. 7, all wavelengths of the visible light are rendered in a pulsating manner. Alternatively, only a (strict) subset of the wavelengths are rendered in a pulsating manner.

As shown in FIGS. 4 to 7, the repairing and/preventing light component is preferably rendered during one or more periods that start at most 24 hours before the UV radiation and end at most 24 hours after the UV radiation.

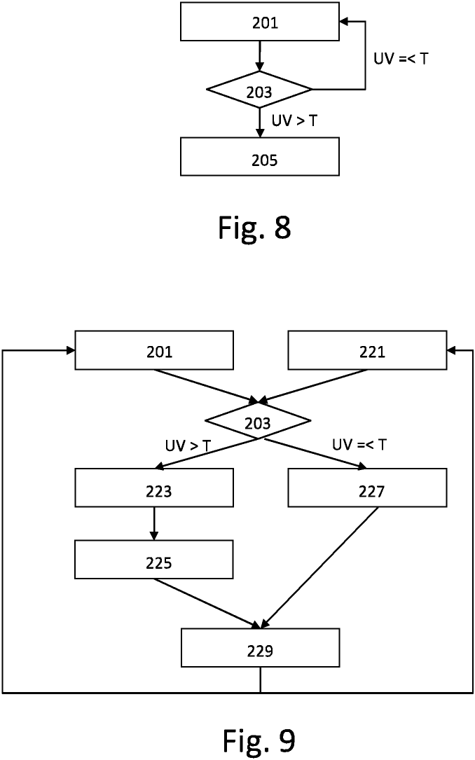

A first embodiment of the method of controlling one or more light sources to render light comprising a light component having wavelengths in the range 550 to 900 nm is shown in FIG. 8. A step 201 comprises determining an amount of UV radiation to which a person has been exposed, an amount of UV radiation to which a person will be exposed or the sum of the these two amounts if applicable. A step 203 comprises determining whether this amount of UV radiation exceeds a threshold T. If it is determined in step 203 that the amount of UV radiation determined in step 201 does not exceed T, then step 201 is repeated at a later time.

If it is determined in step 203, that the amount of UV radiation determined in step 201 does exceed T, then a step 205 is performed. Step 205 comprises controlling the one or more light sources to render light comprising a light component having wavelengths in the range 550 to 900 nm. A spectral power distribution of the light is chosen such that the light has a cytochrome C oxidase efficacy complying with one or more of the following conditions:
(i) the cytochrome C oxidase efficacy for DNA synthesis is at least (6.2*vʹ-2.48) mW/lm if the light’s vʹ is lower than 0.539 or is at least 0.85 mW/lm if the light’s vʹ is equal to or higher than 0.539, and
(ii) the cytochrome C oxidase efficacy for RNA synthesis is at least (7.5*vʹ-2.975) mW/lm if the light’s vʹ is lower than 0.539 or is at least 1.05 mW/lm if the light’s vʹ is equal to or higher than 0.539,
wherein the cytochrome C oxidase efficacy is defined as:

$$\begin{matrix}C y t o c h r o m e C o x i d a s e E f f i c a c y o f r a d i a t i o n \left(W / L m\right) = \\ \frac{{\int }_{550}^{900} {Φ}_{\text{e,} λ} \left(λ\right) {s}_{c y t} \left(λ\right) d λ}{683 {\int }_{380}^{780} {Φ}_{\text{e,} λ} \left(λ\right) V \left(λ\right) d λ}\end{matrix}$$

wherein:
Φ_{e,λ}(λ) = is the spectral power distribution of the light
s_{cyt}(λ) = is the spectral sensitivity of cytochrome C oxidase activation for either DNA synthesis or RNA synthesis;
V(λ) = is the photopic luminosity function; and
vʹ is a color coordinate of the light in the CIE 1976 Uniform Chromaticity Scale diagram.

The term light source may refer to a semiconductor light-emitting device, such as a light emitting diode (LEDs), a resonant cavity light emitting diode (RCLED), a vertical cavity laser diode (VCSELs), or an edge emitting laser. The term light source may also refer to an organic light-emitting diode, such as a passive-matrix (PMOLED) or an active-matrix (AMOLED). In a specific embodiment, the light source comprises a solid state light source (such as a LED or laser diode). In an embodiment, the light source comprises a LED (light emitting diode). The term LED may also refer to a plurality of LEDs.

Further, the term light source may in embodiments also refer to a so-called chips-on-board (COB) light source. The term “COB” especially refers to LED chips in the form of a semiconductor chip that is neither encased nor connected but directly mounted onto a substrate, such as a PCB. Hence, a plurality of semiconductor light sources may be configured on the same substrate. In embodiments, a COB is a multi LED chip configured together as a single lighting module. The term light source may also relate to a plurality of (essentially identical (or different)) light sources, such as 2-2000 solid state light sources. Hence, in embodiments the term “one or more solid state light sources” may also refer to a COB.

In embodiments, the light source may comprise one or more micro-optical elements (array of micro lenses) downstream of a single solid state light source, such as a LED, or downstream of a plurality of solid state light sources (i.e. e.g. shared by multiple LEDs). In embodiments, the light source may comprise a LED with on-chip optics. In embodiments, the light source comprises a pixelated single LEDs (with or without optics) (offering in embodiments on-chip beam steering).

A second embodiment of the method of controlling one or more light sources to render light comprising a light component having wavelengths in the range 550 to 900 nm is shown in FIG. 9. A step 201 comprises determining an amount of UV radiation to which a person has been exposed, an amount of UV radiation to which a person will be exposed or the sum of the these two amounts if applicable. The amount of UV radiation to which a person has been exposed may be the amount of UV radiation received by a light sensor, for example. A step 221 comprises determining a desired color coordinate v′ based on user input.

A step 203 comprises determining whether the amount of UV radiation determined in step 201 exceeds a threshold T. If it is determined in step 203 that the amount of UV radiation determined in step 201 does not exceed threshold T, then step 227 is performed. Step 227 comprises choosing the spectral power distribution of the light to be rendered such that the desired color coordinate v′ is achieved.

If it is determined in step 203, that the amount of UV radiation determined in step 201 does exceed threshold T, then a step 223 is performed. Step 223 comprises determining a minimum target value for the cytochrome C oxidase efficacy based on the desired color coordinate vʹ (determined in step 221) such that the minimum target value is at least 6.2*vʹ-2.48 mW/lm if the desired color coordinate vʹ is lower than 0.539 or at least 0.85 mW/lm if the desired color coordinate vʹ is equal to or higher than 0.539 (if DNA synthesis is desired) and/or the minimum target is at least 7.5*vʹ-2.975 mW/lm if the desired color coordinate vʹ is lower than 0.539 or at least 1.05 mW/lm if the desired color coordinate vʹ is equal to or higher than 0.539 (if RNA synthesis is desired). The minimum target value for the cytochrome C oxidase efficacy, determined in step 223, may further be based on the amount of UV radiation determined in step 201.

Next, a step 225 comprises choosing a spectral power distribution of the light such that the light comprises a light component having wavelengths in the range 550 to 900 nm, the desired color coordinate vʹ is achieved and the cytochrome C oxidase efficacy of the light has a value which equals or exceeds the minimum target value determined in step 223. This may be implemented by first selecting a first spectral power distribution such that the light comprises a light component having wavelengths in the range 550 to 900 nm (or a subset thereof, e.g. 600 to 850 nm) and the desired color coordinate v' is achieved and calculating the cytochrome C oxidase efficacy with the following equation:

$$\begin{matrix}C y t o c h r o m e C o x i d a s e E f f i c a c y o f r a d i a t i o n \left(W / L m\right) = \\ \frac{{\int }_{550}^{900} {Φ}_{\text{e,} λ} \left(λ\right) {s}_{c y t} \left(λ\right) d λ}{683 {\int }_{380}^{780} {Φ}_{\text{e,} λ} \left(λ\right) V \left(λ\right) d λ}\end{matrix}$$

wherein:
Φ_{e,λ}(λ) = is the spectral power distribution of the light
s_{cyt}(λ) = is the spectral sensitivity of cytochrome C oxidase activation for either DNA synthesis or RNA synthesis;
V(λ) = is the photopic luminosity function; and
vʹ is a color coordinate of the light in the CIE 1976 Uniform Chromaticity Scale diagram.

If the cytochrome C oxidase efficacy of the spectral power distribution equals or exceeds the minimum target value determined in step 223, then step a 229 is performed next. If not, then a next spectral power distribution is selected such that the light comprises a light component having wavelengths in the range 550 to 900 nm (or a subset thereof) and the desired color coordinate vʹ is achieved and the cytochrome C oxidase efficacy is then calculated for this next spectral power distribution. This is repeated until a cytochrome C oxidase efficacy is obtained that equals or exceeds the minimum target value determined in step 223.

Step 229 is performed after step 225 or step 227. Step 229 comprises controlling the one or more light sources to render light with spectral power distribution determined in step 225 or step 227. Step 201 and/or step 221 are repeated after step 229 has been performed, after which the method proceeds as shown in FIG. 9.

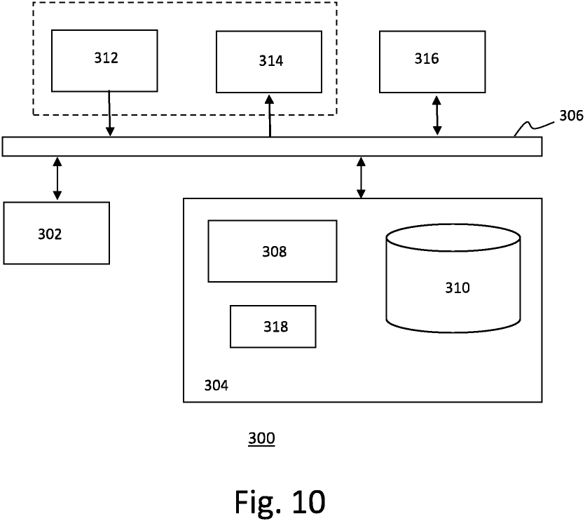

FIG. 10 depicts a block diagram illustrating an exemplary data processing system that may perform the method as described with reference to FIGS. 8 and 9.

As shown in FIG. 10, the data processing system 300 may include at least one processor 302 coupled to memory elements 304 through a system bus 306. As such, the data processing system may store program code within memory elements 304. Further, the processor 302 may execute the program code accessed from the memory elements 304 via a system bus 306. In one aspect, the data processing system may be implemented as a computer that is suitable for storing and/or executing program code. It should be appreciated, however, that the data processing system 300 may be implemented in the form of any system including a processor and a memory that is capable of performing the functions described within this specification.

The memory elements 304 may include one or more physical memory devices such as, for example, local memory 308 and one or more bulk storage devices 310. The local memory may refer to random access memory or other non-persistent memory device(s) generally used during actual execution of the program code. A bulk storage device may be implemented as a hard drive or other persistent data storage device. The processing system 300 may also include one or more cache memories (not shown) that provide temporary storage of at least some program code in order to reduce the quantity of times program code must be retrieved from the bulk storage device 310 during execution. The processing system 300 may also be able to use memory elements of another processing system, e.g. if the processing system 300 is part of a cloud-computing platform.

Input/output (I/O) devices depicted as an input device 312 and an output device 314 optionally can be coupled to the data processing system. Examples of input devices may include, but are not limited to, a keyboard, a pointing device such as a mouse, a microphone (e.g. for voice and/or speech recognition), or the like. Examples of output devices may include, but are not limited to, a monitor or a display, speakers, or the like. Input and/or output devices may be coupled to the data processing system either directly or through intervening I/O controllers.

In an embodiment, the input and the output devices may be implemented as a combined input/output device (illustrated in FIG. 10 with a dashed line surrounding the input device 312 and the output device 314). An example of such a combined device is a touch sensitive display, also sometimes referred to as a “touch screen display” or simply “touch screen”. In such an embodiment, input to the device may be provided by a movement of a physical object, such as e.g. a stylus or a finger of a user, on or near the touch screen display.

A network adapter 316 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the data processing system 300, and a data transmitter for transmitting data from the data processing system 300 to said systems, devices and/or networks. Modems, cable modems, and Ethernet cards are examples of different types of network adapter that may be used with the data processing system 300.

As pictured in FIG. 10, the memory elements 304 may store an application 318. In various embodiments, the application 318 may be stored in the local memory 308, the one or more bulk storage devices 310, or separate from the local memory and the bulk storage devices. It should be appreciated that the data processing system 300 may further execute an operating system (not shown in FIG. 10) that can facilitate execution of the application 318. The application 318, being implemented in the form of executable program code, can be executed by the data processing system 300, e.g., by the processor 302. Responsive to executing the application, the data processing system 300 may be configured to perform one or more operations or method steps described herein.

FIG. 10 shows the input device 312 and the output device 314 as being separate from the network adapter 316. However, additionally or alternatively, input may be received via the network adapter 316 and output be transmitted via the network adapter 316. For example, the data processing system 300 may be a cloud server. In this case, the input may be received from and the output may be transmitted to a user device that acts as a terminal.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 302 described herein.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or hardware components, but do not preclude the presence or addition of one or more other features, integers, steps, operations, elements, hardware components, and/or groups thereof.

The corresponding structures, materials, acts, and equivalents of all means or step plus function elements in the claims below are intended to include any structure, material, or act for performing the function in combination with other claimed elements as specifically claimed. The description of embodiments of the present invention has been presented for purposes of illustration, but is not intended to be exhaustive or limited to the implementations in the form disclosed. Many modifications and variations will be apparent to those of ordinary skill in the art without departing from the scope and spirit of the present invention. The embodiments were chosen and described in order to best explain the principles and some practical applications of the present invention, and to enable others of ordinary skill in the art to understand the present invention for various embodiments with various modifications as are suited to the particular use contemplated.

1. A system for controlling one or more light sources to render light comprising a light component having wavelengths in the range 550 to 900 nm, said system comprising:
at least one control interface; and
at least one processor configured to:
determine whether a person has been and/or will be irradiated with an amount of UV radiation exceeding a threshold, based on at least one of
an amount of UV radiation received by a light sensor, and
UV radiation information from a control signal from a transmitter to the lighting device, and
control, via said at least one control interface, in dependence on said determination, said one or more light sources to render light comprising a light component having wavelengths in the range 550 to 900 nm, a spectral power distribution of said light being chosen such that said light has a cytochrome C oxidase efficacy complying with one or more of the following conditions: the cytochrome C oxidase efficacy for DNA synthesis is at least 6.2*vʹ-2.48 mW/Im if said light’s vʹ is lower than 0.539 or is at least 0.85 mW/Im if said light’s vʹ is equal to or higher than 0.539, and the cytochrome C oxidase efficacy for RNA synthesis is at least 7.5*vʹ-2.975 mW/Im if said light’s vʹ is lower than 0.539 or is at least 1.05 mW/Im if said light’s vʹ is equal to or higher than 0.539, wherein the cytochrome C oxidase efficacy is defined as:
$$\begin{matrix}C y t o c h r o m e C o x i d a s e E f f i c a c y o f r a d i a t i o n \left(W / L m\right) = \\ \frac{{\int }_{550}^{900} {ϕ}_{e λ} \left(λ\right) {S}_{c y t} \left(λ\right) d λ}{683 {\int }_{380}^{722} {ϕ}_{e λ} \left(λ\right) V \left(λ\right) d λ}\end{matrix}$$
wherein:
Φ_{eλ}(λ) = is said spectral power distribution of said light
s_{cyt}(λ) = is the spectral sensitivity of cytochrome C oxidase activation for either DNA synthesis or RNA synthesis;
V(λ) = is the photopic luminosity function; and
vʹ is a color coordinate of said light in the ClE 1976 Uniform Chromaticity Scale diagram.

2. A system as claimed in claim 1, wherein said light component comprises wavelengths in the range 600 to 850 nm.

3. A system as claimed in claim 2, wherein said light component comprises wavelengths in at least one of the ranges: 605 to 635 nm, 660 to 690 nm, 755 to 790 nm and 800 to 835 nm.

4. A system as claimed in claim 1 , wherein said at least one processor is configured to control, via said at least one control interface, said one or more light sources to render said light during one or more periods that start at most 24 hours before said UV radiation and end at most 24 hours after said UV radiation.

5. A system as claimed in claim 1 , wherein said at least one processor is configured to control, via said at least one control interface, said one or more light sources to render said light such that said light includes at least part of said UV radiation, said UV radiation being rendered with a minimum standard erythemal dose of 0.01 per day and a maximum standard erythemal dose of 10 per day.

6. A system as claimed in claim 5, wherein said UV radiation comprises wavelengths in the range 280 to 315 nm and/or wavelengths in the range 315-400 nm.

7. A system as claimed in claim 1 , wherein said at least one processor is configured to:
determine a minimum target value for said cytochrome C oxidase efficacy based on a desired color coordinate vʹ such that said minimum target value for DNA synthesis is at least 6.2*vʹ-2.48 mW/Im if said desired color coordinate vʹ is lower than 0.539 or at least 0.85 mW/Im if said desired color coordinate v' is equal to or higher than 0.539 and said minimum target value for RNA synthesis is at least 7.5*vʹ-2.975 mW/Im if said desired color coordinate vʹ is lower than 0.539 or at least 1.05 mW/Im if said desired color coordinate v' is equal to or higher than 0.539,
choose said spectral power distribution of said light such that said light comprises said light component having wavelengths in the range 550 to 900 nm, said desired color coordinate vʹ is achieved and said cytochrome C oxidase efficacy of said light has a value which equals or exceeds said minimum target value.

8. A system as claimed in claim 7, wherein said at least one processor is configured to determine said minimum target value for said cytochrome C oxidase efficacy further based on said amount of UV radiation.

9. A system as claimed in claim 1 , wherein said light comprises further light components which make said light look white.

10. A system as claimed in claim 1 , wherein said at least one processor is configured to control said one or more light sources to render said light component in a pulsating manner.

11. A system as claimed in claim 1 , wherein said at least one processor is configured to control said one or more light sources to render said light component continuously.

12. A method of controlling one or more light sources to render light comprising a light component having wavelengths in the range 550 to 900 nm, said method comprising:
determining whether a person has been and/or will be irradiated with an amount of UV radiation exceeding a threshold based on at least one of
an amount of UV radiation received by a light sensor,and
UV radiation information from a control signal from a transmitter to the lighting device; and
controlling, in dependence on said determination, said one or more light sources to render light comprising a light component having wavelengths in the range 550 to 900 nm, a spectral power distribution of said light being chosen such that said light has a cytochrome C oxidase efficacy complying with one or more of the following conditions: the cytochrome C oxidase efficacy for DNA synthesis is at least 6.2*vʹ-2.48 mW/Im if said light’s vʹ is lower than 0.539 or is at least 0.85 mW/Im if said light’s vʹ is equal to or higher than 0.539, and the cytochrome C oxidase efficacy for RNA synthesis is at least 7.5*vʹ-2.975 mW/Im if said light’s vʹ is lower than 0.539 or is at least 1.05 mW/Im if said light’s vʹ is equal to or higher than 0.539, wherein the cytochrome C oxidase efficacy is defined as:
$$\begin{matrix}C y t o c h r o m e C o x i d a s e E f f i c a c y o f r a d i a t i o n \left(W / L m\right) = \\ \frac{{\int }_{550}^{900} {ϕ}_{e λ} \left(λ\right) {a}_{c y t} \left(λ\right) d λ}{683 {\int }_{380}^{780} {ϕ}_{e λ} \left(λ\right) V \left(λ\right) d λ}\end{matrix}$$
wherein:
Φ_{eλ}(λ) = is said spectral power distribution of said light
s_{cyt}(λ) = is the spectral sensitivity of cytochrome C oxidase activation for either DNA synthesis or RNA synthesis;
V(λ) = is the photopic luminosity function; and
vʹ is a color coordinate of said light in the CIE 1976 Uniform Chromaticity Scale diagram.

13. A computer program or suite of computer programs comprising at least one software code portion or a computer program product storing at least one software code portion, the software code portion, when run on a computer system, being configured for performing the method of claim 12.
